(19) United States
(12) Patent Application Publication
(10) Pub. No.: US 2006/0162000 A1
(43) Pub. Date: Jul. 20, 2006

(54) Development and use of a new orthotopic, genetically tractable non-human animal model for liver cancer

(76) Inventors: Lars Zender, Cold Spring Harbor, NY (US); Scott W. Lowe, Cold Spring Harbor, NY (US); Mona S. Spector, Cold Spring Harbor, NY (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/325,218

(22) Filed: Jan. 3, 2006

Related U.S. Application Data
(60) Provisional application No. 60/641,043, filed on Jan. 3, 2005.
(60) Provisional application No. 60/686,609, filed on Jun. 1, 2005.

(51) Int. Cl. A01K 67/027; C12N 15/87

(52) U.S. Cl. 800/10; 800/14; 435/455

(57) ABSTRACT

This invention provides a genetically tractable in situ non-human animal model for hepatocellular carcinoma. The model is useful, inter alia, in understanding the molecular mechanisms of liver cancer, in understanding the genetic alterations that lead to chemoresistance or poor prognosis, and in identifying and evaluating new therapies against hepatocellular carcinomas. The liver cancer model of this invention is made by altering hepatocytes to increase oncogene expression, to reduce tumor suppressor gene expression or both and by transplanting the resulting hepatocytes into a recipient non-human animal.

### TECHNICAL FIELD OF THE INVENTION

This invention provides a genetically tractable in situ non-human animal model for liver cancer and specifically hepatocellular carcinoma. The model is useful, inter alia, in understanding the molecular mechanisms of liver cancer, in understanding the genetic alterations that lead to chemoresistance or poor prognosis, and in identifying and evaluating new therapies against hepatocellular carcinomas.

### BACKGROUND INFORMATION

Cancer is the second leading cause of death in industrial countries. More than 70% of all cancer deaths are due to carcinomas, i.e., cancers of epithelial organs. Most carcinoma tumors show initial or compulsory chemoresistance. This property makes it very difficult to cure these tumors when they are detected in progressed stages. Primary forms of liver cancers include hepatocellular carcinoma, biliary tract cancer and hepatoblastoma. Hepatocellular carcinoma is the fifth most common cancer worldwide but, owing to the lack of effective treatment options, constitutes the leading cause of cancer deaths in Asia and Africa and the third leading cause of cancer death worldwide. Parkin et al. “Estimating the world cancer burden: Globocan 2000.” Int. J. Cancer 94, 153-156 (2001).

The risk factors for liver cancer include excessive alcohol intake or other toxins, such as iron, aflatoxin B1 and also the presence of other infections such as hepatitis B and C. Alison & Lovell. “Liver cancer: the role of stem cells.” Cell Prolif 38, 407-421 (2005). The only curative treatments for hepatocellular carcinoma are surgical resection or liver transplantation, but most patients present with advanced disease and are not candidates for surgery. To date, systemic chemotherapeutic treatment is ineffective against hepatocellular carcinoma, and no single drug or drug combination prolongs survival. Llovet. et al. “Hepatocellular carcinoma.” Lancet 362, 1907-1917. (2003). However, despite its clinical significance, liver cancer is understudied relative to other major cancers.

One of the difficulties in identifying appropriate therapeutics for tumor cells in vivo is the limited availability of appropriate test material. Human tumor lines grown as xenographs are unphysiological, and the wide variation between human individuals, not to mention treatment protocols, makes clinical studies difficult. Consequently, oncologists are often forced to perform correlative studies with a limited number of highly dissimilar samples, which can lead to confusing and unhelpful results.

Non-human animal models provide a useful alternative to studies in humans and to human tumor cell lines grown as xenographs, as large numbers of genetically-identical individuals can be treated with identical regimens. Moreover, the ability to introduce germline mutations that affect oncogenesis into these animals increases the power of the models.

To investigate the basic mechanisms of carcinogenesis and to test new potential cancer agents and therapies, however, realistic carcinoma-non-human animal models are urgently needed. So far there have been two major ways to create carcinoma non-human animal models: (i) the generation of transgenic or chimeric non-human animals that express oncogenes under the control of a tissue specific promoter and (ii) carcinomas that were induced by chemical carcinogens. Both approaches have several disadvantages.

Current animal models for cancer are based largely on classical transgenic approaches that direct expression of a particular oncogene to an organ of choice using a tissue specific promoter. See, e.g., Wang et al. “Activation of the Met receptor by cell attachment induces and sustains hepatocellular carcinomas in transgenic mice.” J. Cell Biol. 153, 1023-1034 (2001). Although such models have provided important insights into the pathogenesis of cancer, they express the active oncogene throughout the entire organ, a situation that does not mimic spontaneous tumorigenesis. Moreover, incorporation of additional lesions, such as a second oncogene or loss of a tumor suppressor, requires genetic crosses that are time consuming and expensive, and again produce whole tissues that are genetically altered. Finally, traditional transgenic and knockout strategies do not specifically target liver progenitor cells, which may be the relevant initiators of the disease.

Cancer therapies that directly target oncogenes are based on the premise that cancer cells require continuous oncogenic signaling for survival and proliferation. Non-human animal models expressing oncogenes in genetic backgrounds that lack, or have down-regulated, tumor suppressor genes can thus serve as valuable tools to study tumor initiation, maintenance, progression, treatment and regression. However, responses to the targeting drugs are often heterogeneous, and chemoresistance and other resistance is a problem. Because most anticancer agents were discovered through empirical screens, efforts to overcome resistance are hindered by a limited understanding of why these agents are effective and when and how they become less or non-effective.

Variations in both non-human animal strains and promoters used to drive expression of oncogenes complicate the interpretation of cancer mechanistics and treatment analyses. Firstly, intercrossing strategies to obtain non-human animals of the desired genetic constellation are extremely time consuming and costly. Secondly, the use of certain cell-selective promoters can result in a cell-bias for tumor initiation. For example, the mouse mammary tumor virus (MMTV) promoter and the Whey Acidic Protein (WAP) promoter are commonly used to model breast cancer development in mice, and yet may not target all subtypes of mammary epithelia, i.e., stem cell and non-stem cells.

An additional difficulty in identifying and evaluating the efficacy of cancer agents on tumor cells and understanding the molecular mechanisms of the cancers and their treatment in the current non-human animal models in vivo is the limited availability of appropriate material. A homogenous expression of the respective oncogene in all epithelial cells of an organ creates an unphysiological condition, as tumors are known to originate within genetic-mosaics. It is therefore important to use a valid model to target distinct genetic pathways and to identify new therapeutics for the treatment of liver cancer.

### SUMMARY OF THE INVENTION

The invention provides in vivo and in vitro systems and methods for the study of the effects of tumorigenesis, tumor maintenance, tumor regression and altered expression of a gene activity, on the descendants of embryonic liver progenitor cells, or primary hepatocytes, that have been engineered to produce hepatocellular carcinomas.

The liver cancer model of this invention is made by altering hepatocytes to increase oncogene expression, to reduce tumor suppressor gene expression or both and by transplanting the resulting hepatocytes into a recipient non-human animal. The spontaneous mutations arising in tumors initiated by different oncogenic lesions are compared to alterations observed in human cancers. Preferably, the transplanting is carried out so that the hepatocytes engraft the liver of the animal and a liver cancer tumor develops there from at least one of the altered hepatocytes. Less preferably, the altered hepatocytes are transplanted subcutaneously into a non-human animal so as to develop a tumor.

The non-human animal model of hepatocellular carcinoma embodied herein is useful for identifying molecular targets for drug screening, for identifying interacting gene activities, for identifying therapeutic treatments and for identifying candidates for new therapeutic treatments. The invention also provides methods and non-human animals produced by the methods that are useful for understanding liver cancer and its treatments, and in particular, for identifying and studying inhibitors and activators associated with liver tumor cell growth and growth inhibition, cell death through apoptotic pathways, and changes in apoptotic pathway components that affect drug sensitivity and resistance in tumorigenic cells.

The genetically tractable, transplantable in situ liver cancer model of this invention is characterized by genetically defined hepatocellular carcinomas that are preferably traceable by external green fluorescent protein (GFP) imaging. To further characterize the genetic defects in these tumors, gene expression profiling, e.g., representational oligonucleotide microarray analysis (ROMA), can be used to scan the carcinomas for spontaneous gains and losses in gene copy number. Detecting genomic copy number changes through such high resolution techniques can be useful to identify oncogenes (amplifications or gains) or tumor suppressor genes (deletions or losses). Identification of overlapping genomic regions altered in both human and mouse gene array datasets may further aid in pinpointing of regions of interest that can be further characterized for alterations in RNA and protein expression to identify candidates are most likely to contribute to the disease phenotype and to be the “driver gene” for amplification.

Using “forward genetics” in combination with gene expression profiling (e.g., ROMA) and the non-human animal models of this invention, important insights into the molecular mechanisms of hepatocarcinogenesis, growth, maintenance, regression and remission can be obtained. The models of the invention can directly evaluate the potency of various oncogenes in producing anti-apoptotic phenotypes, and various tumor suppressor genes in producing apoptotic phenotypes. Candidate oncogenes or tumor suppressors can be rapidly validated in the mouse model of the invention by overexpression, or by using stable RNAi technology, respectively. The invention is also useful in analyzing and evaluating genetic constellations that confer chemoresistance or poor prognosis. Furthermore, the invention is useful for identifying and evaluating new therapies for the treatment of carcinomas.

### BRIEF DESCRIPTION OF THE DRAWINGS

FIG. 1: Development and characterization of a new orthotopic, genetically tractable mouse model for hepatocellular carcinoma. (a) Schematic outline of two claims of producing a non-human animal liver cancer model of this invention. E-Cadherin+mouse hepatoblasts are isolated from day 13-15 mouse liver using the MACS® indirect labeling system in combination with the ECCD-1 E-Cadherin antibody. Purified hepatoblasts are grown in short term primary-culture on irradiated NIH-3T3 feeder layers. The hepatoblasts are infected with GFP-tagged murine stem cell virus (MSCV) based retroviruses expressing oncogenes of interest (e.g., the H-ras oncogene) and/or expression cassettes for short hairpin RNAs directed against tumor suppressor genes (e.g., p53). After viral transduction, infected hepatoblasts are either injected into the spleens of retrorsine-conditioned recipient mice or subcutaneously into NCR nu/nu mice. Retrorsine efficiently blocks the cell cycle of hepatocytes and additionally causes a moderate liver damage by triggering apoptosis in a small number of hepatoblasts. Using this approach, after intrasplenic transplantation, genetically modified hepatocytes migrate via the portal vein into the recipient liver and engraft the organ. Transplanted hepatoblasts harboring the defined genetic lesions clonally expand and hepatocellular carcinomas develop in the liver. Tumor onset and growth kinetics can be monitored by external whole body GFP-imaging as all viral vectors carry a GFP expression cassette. (b) Transplanted hepatoblasts engraft the recipient liver and are morphologically indistinguishable from the host hepatocytes (H&E). Immunofluorescence with a primary antibody directed against GFP allows detection of the transplanted hepatocytes (middle). DAPI counterstaining (right). (c) p53 deficient liver progenitor cells transduced with different oncogenes (myc, akt or H-rasV12) give rise to orthotopic liver carcinomas after intrahepatic seeding. Detection of intrahepatic liver carcinomas by whole body, external GFP-tumor imaging (top panel) or direct imaging of the respective explanted tumor bearing livers (bottom panel). Tumors can be detected by either external GFP imaging (upper panel) or direct GFP-imaging of the explanted liver (lower panel). (d) Kaplan-Meier curve for survival times of mice transduced with different oncogenes (myc, akt, H-rasV12). All groups succumb to death much earlier than mice injected with p53−/− control vector alone. (e) Cells from primary murine liver carcinomas (p53−/−; myc) were grown briefly in culture. In situ liver carcinomas were generated by direct subcapsular injection of the cells into the left liver lobe. Shown are GFP-tumor imaging (left) and a photograph of the in situ tumors (right) day 42 post-injection.

FIG. 2: Genome-wide analysis of copy number alterations in mouse hepatocellular carcinoma (HCC). DNA from tumors and subjected to 85K ROMA. Plotted is the normalized log-ratio for each oligo probe and ordered according to genome position, derived from the May 2004 freeze of the draft mouse genome sequence (http://www.genome.ucsc.edu). (a) Representative profiles of 3 mouse HCCs. HCC-7 and HCC-9, both derived from p53−/−; c-myc hepatoblasts, contain an amplification on chromosome 9. HCC-11, derived from p53−/−; Akt hepatoblasts, does not. (b) Expanded view of chromosome 9 reveals a 1.9 Mb amplicon (HCC-9) and a 1.2 Mb (HCC-7) amplicon containing the c-IAP-1 and c-IAP-2 genes. (c) Quantitative PCR with primers specific for the c-IAP-1 gene revealed higher copy numbers for 2 additional p53−/−; c-myc HCCs (HCC-13 and HCC-14), while non-c-myc tumors (HCC-15 and HCC-17) have a normal IAP copy number. (d) Summary of c-IAP-1/2 amplification relative to genetic background. (e) c-IAP-1 and c-IAP-2 mRNA levels are elevated in tumors containing the amplicon. Levels of IAP RNA relative to actin were determined by quantitative RT-PCR and normalized to normal liver.

FIG. 3: Genome-wide analysis of a human hepatocellular carcinoma analyzed with 36K ROMA. (a) The three peaks indicate amplicons containing the MET-oncogene, Cyclin D and c-IAP1/2 (left to right). (b) Expanded view of chromosome 11 showing the amplicons containing cyclin D and c-IAP1/2. (c) 1/25 human HCCs have elevated c-IAP-1 and c-IAP-2 gene copy numbers as determined by quantitative PCR off genomic DNA. (d) c-IAP-1 and/or c-IAP-2 mRNA levels are elevated in 4/25 HCCs as determined by quantitative RT-PCR.

FIG. 4: c-IAP-1 overexpression accelerates tumor growth. (a) Schema of the approach to investigate the oncogenic properties of c-IAP-1 in vivo. E-Cadherin+hepatoblasts were either double-infected with c-myc+control vector or c-myc+myc-tag-c-IAP-1. 10×106 cells were subcutaneously injected into irradiated NCR nu/nu mice. (b) Overexpression of c-IAP-1 in primary liver cells was confirmed by western blot analysis using an α-myc-tag antibody. (c) Four out of six c-myc+c-IAP-1 double infected tumors show accelerated tumor growth compared to c-myc+vector. Tumor size was assessed by caliper measurement of subcutaneously growing tumors. (d) All tumors showing accelerated growth contain the c-IAP-1 provirus as assayed by PCR. All analyzed tumors contain c-myc-provirus DNA. (e) Representative example of an accelerated c-IAP1+c-myc double infected tumor (left) compared to a c-myc+vector infected tumor (right). External GFP-imaging of the tumors (bottom) was performed at the same time post-injection.

FIG. 5: Suppression of c-IAP-1 in HCC cells slows tumor growth. (a) Schema for testing knock-down of c-IAP-1 expression in vivo. Cells from tumors containing the c-IAP1/2 amplicon are outgrown briefly and infected either with a retrovirus expressing a short hairpin (miR30 design) RNA directed against c-IAP-1 or with control vector. After puromycin-selection, cells were injected subcutaneously into NCR nu/nu mice. (b) One out of four short hairpins directed against c-IAP-1 suppresses c-IAP-1 expression. NIH 3T3 cells were transiently transfected with pcDNA-myc tag-c-IAP-1 together with the respective hairpin. Western blot was performed using an α-myc-tag antibody. c-IAP-1-hairpin “1477” shows >95% knockdown (c) Tumors with stable RNAi mediated knockdown of c-IAP-1 show decelerated tumor growth compared to control vector infected tumors. Growth of subcutaneous tumors was assesed by caliper measurement. (d) Representative example of a slower growing tumor with c-IAP-1 knockdown (right) compared to a control vector infected tumor. External GFP-imaging of the tumors (top panel) was done at the same time post-injection.

FIG. 6: Influence of c-IAP-1 overexpression on proliferation and apoptosis in cultured hepatoblasts. (a) ECadherin+hepatoblasts were infected with a neomycin selectable retrovirus overexpressing c-myc and a puromycin selectable retrovirus overexpressing c-IAP-1 or control vector. After neomycin/puromycin selection cells were plated at 4.5×103 cells/cm2 and growth rate was assessed by daily counting of the total cell number. c-IAP-1 overexpressing cells have a slight growth advantage. (b) c-myc+c-IAP-1 or c-myc+vector double infected

FIG. 7: An example of subcutaneous liver cancer model of this invention on the genetic constellation p53−/−+Akt-over expression and its uses in evaluating tumor therapy. Akt is an apoptotic regulator that is activated in many cancers and may promote drug resistance in vitro (Mayo et al., “PTEN protects p53 from Mdm2 and sensitizes cancer cells to chemotherapy,” J. Biol. Chem. 277: 5484-5489 (2002)). The graph shows that the tumor's intrinsic chemoresistance against the cancer drug Gemcitabine® (brand name “Gemzar” in the Figure) can be reversed by application of a downstream effector of Akt, the mTOR (mammalian target of rapamycin) inhibitor Rapamycin.

FIG. 8: ROMA identifies localized DNA amplifications in murine HCCs. (a) Genome-wide profile of a tumor derived from p53−/−; ras embryonic hepatoblasts reveals 2 amplifications on chromosome 15. Data plotted is the normalized log ratio for each probe (85K), sorted by chromosome position, of copy number for tumors relative to a normal reference DNA. (b) Single probe resolution of chromosome 15 reveals increased copy number for Rnf19 and myc. (c) Genome-wide profiles of three independent HCCs (Tu-7, Tu-9, Tu-13) derived from p53−/−; myc embryonic hepatoblasts are overlaid and reveal a recurrent overlapping DNA amplification on chromosome 9. (d) Single probe resolution of the amplicon on chromosome 9qA1; minimal overlap region contains genes indicated.

FIG. 9: ROMA identifies amplification of the human syntenic region 11q22 in HCC and other cancers. (a) Genome-wide profile of a human HCC reveals an amplification on chromosome 7 containing the c-MET gene and 3 regions amplified on chromosome 11. (b) Single probe resolution of chromosome 11; 11q13 contains CCND1; B′ contains no known genes; 11 q22 contains the genes depicted. (c) Single probe resolution of chromosome 11 of a representative esophageal tumor. 11 q22 contains same genes as shown in b. (d) Genome-wide profile of an ovarian carcinoma reveals chromosome 11 amplification. (e) Single probe resolution of the 11q22 amplicon shows a lack of amplification of the MMP cluster.

FIG. 10: c1AP1 is overexpressed in tumors containing elevated c1AP1 gene copy number (a) c1AP1 and c1AP2 are overexpressed in murine HCCs as determined by quantitative realtime RT-PCR analysis. *denotes tumors with elevated c1AP1 gene copy number. (b) c1AP1 protein is overexpressed in outgrown murine HCC tumor cells containing the 9qA1 amplicon as assayed by immunoblotting using a monoclonal anti-c1AP antibody (top panel) or a polyclonal anti-c1AP1/2 antiserum (middle panel). Tubulin served as a loading control. (c) Quantitative real-time RT-PCR analysis of c1AP1 and c1AP2 expression in human HCCs. (d) Quantitative real-time RT-PCR analysis of c1AP1 and c1AP2 expression in a subset of human esophageal carcinomas. *denotes tumor with elevated c1AP1 gene copy number (c+d).

FIG. 11. c1AP1 overexpression in p53−/−; myc hepatoblasts suppresses p53 independent forms of apoptosis induced by different death stimuli. (a) Expression of myc-tagged-c1AP1 in p53−/−; myc liver progenitor cells (right lane) was confirmed by western blot analysis using a monoclonal anti-c1AP1 antibody. Left lane (V) is lysate from cells infected with vector alone. (b) c1AP1 expression protects hepatoblasts from apoptosis mediated by serum withdrawal. p53−/− hepatoblasts, double infected with myc+c1AP1 or myc+vector were grown in the serum conditions indicated for 48 hrs. Apoptosis was measured using the Cell Death Detection ELISAPLUS (Roche). Error bars denote the standard deviation of three measurements per data point (B-D). (c) c1AP1 protects against spontaneous cell death mediated by contact inhibition. p53−/− hepatoblasts (myc+c1AP1 or myc+vector) were grown to confluence and apoptosis was measured [as in (B)] 24 hours later. (d) c1AP1 protects against FasL triggered cell death but not TNFa or TRAIL mediated cell death of liver progenitor cells. p53−/− hepatoblasts (myc+c1AP1 or myc+vector) were treated with 125 ng/ml TRAIL, 5 ng/ml TNFa or increasing concentrations of FasL (25, 50, 100 ng/ml) together with 2.5 ug/ml cycloheximide for 12 hrs. Apoptosis was measured as in (b). (e) c1AP1 increases short and long-term viability following FasL treatment. p53−/− hepatoblasts (Myc+c1AP1 or Myc+vector) were treated with 50 ng/ml FasL for 36 hrs. Representative phase contrast photographs (upper panel, left) depict floating, apoptotic cells (arrow) and viable, attached cells (right, arrowhead). Cells that underwent the same treatment were cultured for 5 more days without FasL and stained with crystal violet to visualize clonogenic survival (lower panel).

FIG. 12. c1AP1 enhances the tumorigenicity of Myc overexpressing p53−/− hepatoblasts. (a) p53−/− hepatoblasts were double-infected with myc plus myc-tagged-c1AP1 or myc plus vector and were subcutaneously injected into the rear flanks of nude mice (n=6 for each group). Tumor size was assesed by caliper measurement. Shown is a representative of three independent experiments. (b) Immunoblot of tumor lysates for c1AP1 protein level. Protein lysates from 6 representative tumors overexpressing myc-tagged-c1AP1 (lanes 8-13) and control vector tumors (lanes 5-7) were probed with anti-c1AP1 antibody. Cells lysates from cultured myc-tagged-c1AP1 expressing hepatoblasts (M, lane 2) or vector alone (V, lane 1) and from 9qA1 amplicon containing cells (A+, lane 4) were loaded for comparison to indicate exogenously expressed, myc-tagged-c1AP1 (75 kD) and endogenous c1AP1 (65 kD). A− is lysate from amplicon negative cells of the same genotype (p53−/−; myc). Tubulin is used as a loading control. (c) c1AP1 does not enhance the tumorigenicity of H-rasV12 overexpressing p53−/− hepatoblasts. n=6 for each group. (d) Immunoblot of tumor lysates from 3 representative mice of each group shown in (C) probed with anti-c1AP1 antibody. (e) c1AP1 does not enhance the tumorigenicity of Akt overexpressing p53−/− hepatoblasts. (f) Immunoblot of tumor lysates from 3 representative mice of each group shown in (e) probed with anti-c1AP1 antibody.

FIG. 13. Tumors bearing the 9qA1 amplicon show delayed growth upon c1AP1 and c1AP2 suppression. (a) Hepatoma cells outgrown from a 9qA1 amplicon positive, p53−/−; Myc tumor, were double infected with shRNAs targeting c1AP1 and c1AP2 or control vectors (V), or no vector (−). Expression of c1AP1 and c1AP2 is significantly reduced as shown on the immunoblots that were probed with a monoclonal anti-c1AP1 antibody (top panel) and a polyclonal anti-c1AP1/2 antibody (second panel). The levels of XIAP were not reduced (third panel). Equal amounts of protein were loaded as assessed by tubulin levels. *denotes a non-specific band. (b) Stable suppression of c1AP1 and c1AP2 slows tumor growth of p53−/−; myc mouse hepatoma cells that contain the 9qA1 amplicon. Tumorigenicity of the cells described in (a) after injection into the rear flanks of nude mice. Growth of subcutaneous tumors was assessed by caliper measurement. (c) Stable suppression of c1AP1 and c1AP2 does not slow tumor growth of p53−/−; myc mouse hepatoma cells that do not contain the 9qA1 amplicon. (d) Stable suppression of p53 does not slow tumor growth of p53−/−; myc mouse hepatoma cells that contain the 9qA1 amplicon.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The methods and techniques of the present invention are generally performed according to conventional methods well known in the art and as described in various general and more specific references that are cited and discussed throughout the present specification, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2003); Harlow and Lane, Antibodies: A Laboratory Manual, Cold Spring Harbor Laboratory Press, Cold Spring Harbor, N.Y. (1990); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W.H. Freeman & Co., New York (2000); Griffiths et al., Introduction to Genetic Analysis, 7th ed., W.H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All of the above and any other publications, patents and published patent applications referred to in this application are specifically incorporated by reference herein.

The genetically tractable, transplantable in situ liver or hepatocellular cancer model of the invention offers unique advantages. This invention employs the proliferative capacity of the liver to enable the altered hepatocytes to reconstitute liver tissue. Large amounts of primary epithelial cells can be isolated according to standardized protocols either from adult mouse livers or from embryonic mouse livers. The primary culture conditions for embryonic, as well as adult primary hepatocytes, are based on well-established protocols and are less complex compared to other epithelial primary cultures. A sample of the primary cells can be used for RT-PCR characterization for liver specific markers to rule out overgrowing by non-parenchymal cells.

Primary adult or embryonic hepatocyte cultures can be genetically modified by infection with lentiviral- or retroviral vectors carrying various genetic alterations, including oncogenes or short hairpin RNAs against tumor suppressor genes. Virally transduced primary hepatocytes can efficiently engraft the livers of non-human animals after transplantation into their portal vein or spleen. In the case of certain genetic configurations, mice developed hepatocellular carcinomas that could be visualized by whole body fluorescence imaging. For example, introduction of a myc retrovirus into p53 deficient hepatocytes produced highly aggressive tumors that show many features of human hepatocellular carcinoma. Overall, it provides rapid generation of genetically defined hepatocellular carcinomas.

The invention embodies a method of making a non-human animal bearing a liver cancer using transplanted hepatocytes altered to increase oncogene expression, to reduce tumor suppressor gene expression or both. Preferably, the hepatocytes are virally transduced with a vector expressing an oncogene or a short hairpin RNA against a tumor suppressor gene and subsequently transplanted into a recipient non-human animal wherein the animal develops liver cancer tumors from at least one of the hepatocytes with altered gene expression.

As used herein, a non-human animal includes any animal, other than a human. Examples of such non-human animals include without limitation: aquatic animals, e.g., fish, sharks, dolphins and the like; farm animals, e.g., pigs, goats, cows, horses, rabbits and the like; rodents, e.g., rats, guinea pigs and mice; non-human primates, e.g., baboons, chimpanzees and monkeys; and domestic animals, e.g., cats and dogs. Rodents are preferred. Mice are more preferred.

The non-human animals can be wild type or can carry genetic alterations. For example, they may be immunocompromised or immunodeficient, e.g., a severe combined immunodeficiency (SCID) animal.

As used herein, hepatocytes include all descendants of embryonic liver progenitor cells. Preferably, primary hepatocytes are used in the methods and models of this invention. Primary hepatocytes from adult non-human animals or embryonic liver progenitor cells can be isolated using standard and conventional protocols. In short term primary culture the hepatocytes can be virally transduced with vectors carrying oncogenes and/or expression cassettes for short hairpin RNAs directed against tumor suppressor genes. Such transductions may be effected using standard and conventional protocols.

The term vector refers to a nucleic acid molecule capable of transporting another nucleic acid to which it has been linked. One type of vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in a host cell into which they are introduced (e.g., vectors having an origin of replication which functions in the host cell). Other vectors can be integrated stably into the genome of a host cell upon introduction into the host cell, and are thereby replicated along with the host genome. Preferred viral vectors include retroviral and lentiviral vectors. Moreover, certain preferred vectors are capable of directing the expression of nucleic acid sequences to which they are operatively linked. Such vectors are referred to herein as recombinant expression vectors or simply, expression vectors. Preferably, the vector carries marker cassettes, more preferably, GFP expression cassettes, so that the course of transduction, engrafting and tumor growth and remission may be observed. Preferably, the vector also carries a ubiquitous promoter to permit expression or up-regulation of oncogenes in all cell types of epithelium (i.e., stem cell and non-stem cell compartments).

As used herein, viral transduction refers to a general method of gene transfer. As embodied herein, viral transduction is used for establishing stable expression of genes in culture. Viral transduction and long-term expression of genes in cells, preferably cultured hepatocytes, is preferably accomplished using viral vectors.

After viral transduction the cells are preferably injected into the spleen of the recipient non-human animal, preferably a rodent and most preferably a mouse, that are preferably pretreated with a liver cell cycle inhibitor. Using this approach, the genetically modified or altered hepatocytes migrate via the portal vein into the recipient liver and engraft the organ. An additional proliferation stimulus to the liver can preferably be given after hepatocyte transplantation by serial administration of CCl₄.

Non-human animals harboring hepatocellular carcinomas of different genetic constellations produced by the altered hepatocytes can be characterized with regard to time to tumor onset and survival time. Tumors of different genetic constellations can also be histologically examined and classified by experienced pathologists.

As used herein, an altered hepatocyte refers to a change in the level of a gene and/or gene product with respect to any one of its measurable activities in a hepatocyte (e.g., the function which it performs and the way in which it does so, including chemical or structural differences and/or differences in binding or association with other factors). An altered hepatocyte may be effected by one or more structural changes to the nucleic acid or polypeptide sequence, a chemical modification, an altered association with itself or another cellular component or an altered subcellular localization. Preferably, an altered hepatocyte may have “activated” or “increased” expression of an oncogene, “repressed” or “decreased” expression of a tumor suppressor gene or both.

The increased expression of an oncogene refers to a produced level of transcription and/or translation of a nucleic acid or protein product encoded by an oncogenic sequence in a cell. Increased expression or up regulation of an oncogene can be non-regulated (i.e., a constitutive “on” signal) or regulated (i.e., the “on” signal is induced or repressed by another signal or molecule within the cell). An activated oncogene can result from, e.g., over expression of an encoding nucleic acid, an altered structure (e.g., primary amino acid changes or post-transcriptional modifications such as phosphorylation) which causes higher levels of activity, a modification which causes higher levels of activity through association with other molecules in the cell (e.g., attachment of a targeting domain) and the like.

The decreased expression of a tumor suppressor gene refers to an inhibited, inactivated or down regulated level of transcription and/or translation of a nucleic acid or protein product encoded by a tumor suppressor gene sequence in a cell. Reduced expression of a tumor suppressor gene can be non-regulated (i.e., a constitutive “off” signal) or regulated (i.e., the “off” signal is activated or repressed by another signal or molecule within the cell). As preferred herein, a repressed tumor suppressor gene can result from inhibited expression of an encoding nucleic acid (e.g., most preferably a short hairpin RNA using RNA interference approaches, see supra). Reduced expression of a tumor suppressor gene can also result from an altered structure (e.g., primary amino acid changes or post-transcriptional modifications such as phosphorylation) which causes reduced levels of activity, a modification which causes reduced levels of activity through association with other molecules in the cell (e.g., binding proteins which inhibit activity or sequestration) and the like.

A short hairpin RNA refers to a segment of RNA that is complementary with a portion of one or more target genes (i.e. complementary with one or more transcripts of one or more target genes). When a nucleic acid construct encoding a short hairpin RNA is introduced into a cell, the cell incurs partial or complete loss of expression of the target gene. In this way, a short hairpin RNA functions as a sequence specific expression inhibitor or modulator in transfected cells. The use of short hairpin RNAs facilitates the down-regulation of tumor suppressor genes and allows for analysis of hypomorphic alleles. The short hairpin RNAs that are useful in the invention can be produced using a wide variety of RNA interference (“RNAi”) techniques that are well known in the art. The invention may be practiced using short hairpin RNAs that are synthetically produced as well as microRNA (miRNA) molecules that are found in nature and can be remodeled to function as synthetic silencing short hairpin RNAs. A preferred claim of the invention is the use of a short hairpin RNA that mediates inhibition of a oncogenic signal, preferably a tumor suppressor gene and thus apoptotic signaling in a cell. Preferably, the short hairpin RNAs are against c1AP1 and c1AP2.

Other methods of RNA interference may also be used in the practice of this invention. See, e.g., Scherer and Rossi, Nature Biotechnology 21:1457-65 (2003) for a review on sequence-specific mRNA knockdown of using antisense oligonucleotides, ribozymes, DNAzymes, RNAi and siRNAs. See also, International Patent Application PCT/US2003/030901 (Publication No. WO 2004/029219 A2), filed Sep. 29, 2003 and entitled “Cell-based RNA Interference and Related Methods and Compositions”.

As used herein the term liver or hepatocellular cancer tumor refers to a group of cells which are committed to a hepatocellular lineage and which exhibit an altered growth phenotype. The term encompasses tumors that are associated with hepatocellular malignancy (i.e., HCC) as well as with pre-malignant conditions such as hepatoproliferative and hepatocellular hyperplasia and hepatocellular adenoma, which include proliferative lesions that are perceived to be secondary responses to degenerative changes in the liver.

The non-human animals of the invention are useful in the study of the impact of genotype on pathology or treatment response in vivo. Thus, the methods and models of the invention have implications for understanding disease progression in human liver carcinomas of specific genetic origin. The invention is also useful for determining the efficacy of a therapy in treating liver cancer. For example, a potential therapy may be administered to a non-human animal, produced by the methods embodied herein, and the non-human animal monitored for liver tumor formation, growth, progression or remission. Often, increased time to tumor formation or growth indicates sensitivity of the tumor to the therapy.

Genomic analysis of human carcinomas can be performed by gene expression profiling, e.g., ROMA. Such analysis in the tumors produced according to the invention has revealed a low signal to noise ratio of profiled genes, suggesting that the majority of detected genetic alterations in human tumors (having a high signal to noise ratio) may not be originally involved in tumor development but may be a by-product of tumor development. The analysis of mouse tumors produced according to the invention has shown that these tumors have a low signal to noise ratio, suggesting that a higher proportion of the identified lesions are specifically involved in tumor initiation/progression. Thus, the analysis of mouse tumors by gene expression profiling can serve as a filter for the “noisy” human tumors. Results obtained from mouse profiling using ROMA can be aligned with ROMA data obtained from human hepatocellular carcinomas. Overlapping amplifications or deletions then can be prioritized for further evaluation.

Tumors showing specific amplifications of candidate oncogenes in gene expression profiles can be outgrown in culture. Using stable RNAi, efficient knockdown of these genes can be achieved. Tumor cells with stable knockdown of a previously amplified gene can be re-transplanted into the mouse model of the current invention. Using this approach new therapeutic targets for hepatocellular carcinoma and related carcinomas can be obtained and the specific consequences of knocking down an amplified gene with regard to tumor growth or metastases can be studied. Drug therapies that specifically inhibit the identified targets can be developed.

Therapies that may be tested and evaluated in the methods and models of this invention include both general and targeted therapies. As used herein, a general therapy can be, for example, a pharmaceutical or chemical with physiological effects, such as pharmaceuticals that have been used in chemotherapy for cancer. Chemotherapeutic agents inhibit proliferation of tumor cells, and generally interfere with DNA replication or cellular metabolism. See, e.g., The McGraw-Hill Dictionary of Chemical Terms (Parker, S., Ed., McGraw-Hill, San Francisco (1985)). Chemotherapeutic agents may or may not have been characterized for their target of action in cells. However, this invention and its methods and models allow evaluation of such therapies for defined genetic alterations.

A targeted therapy refers to a therapy that directly interferes with a specific gene Preferably, a targeted therapy directly interferes with the expression of a gene involved in liver cancer. The effectiveness of a targeted therapy can be determined by the ability of the therapy to inhibit an oncogene or activate a tumor suppressor gene.

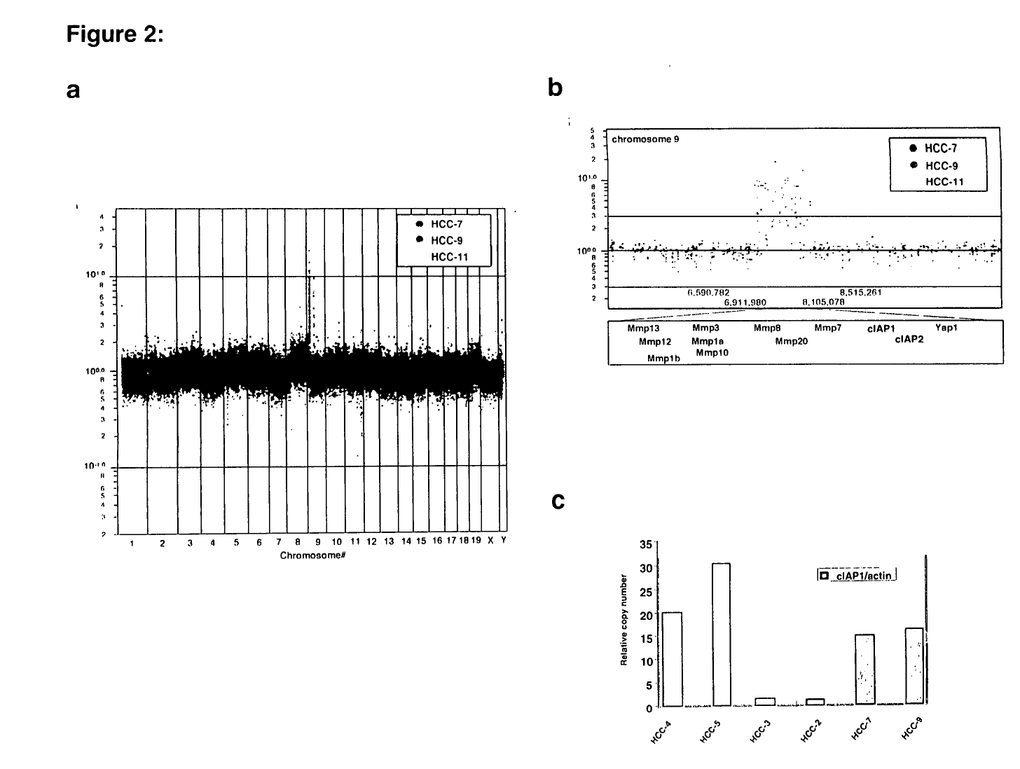

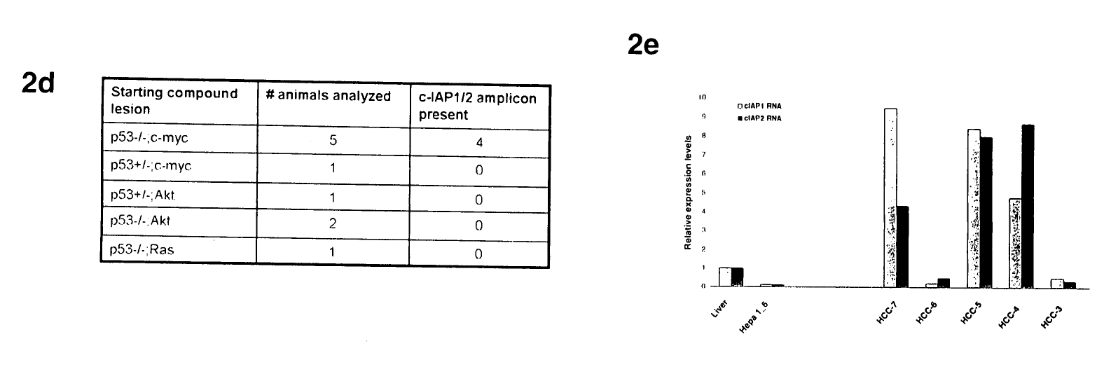

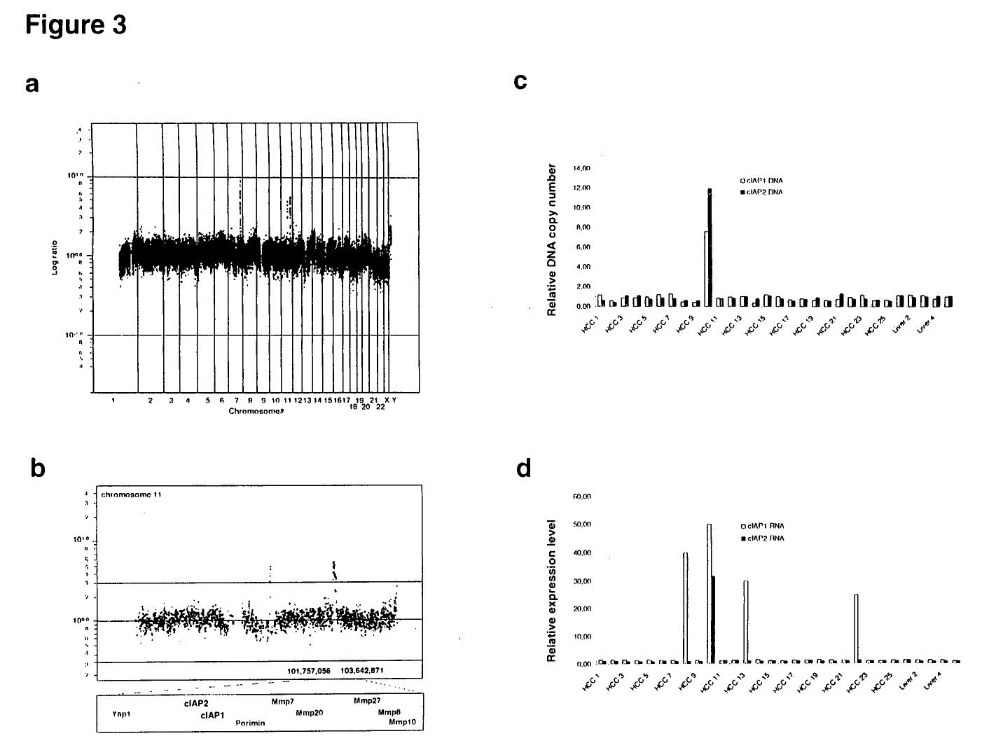

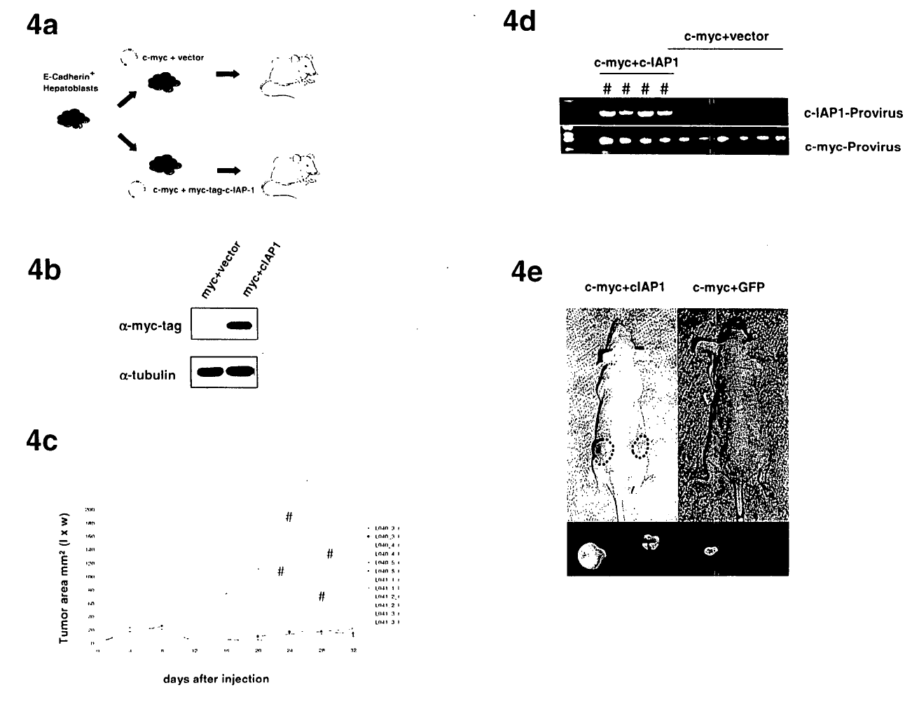

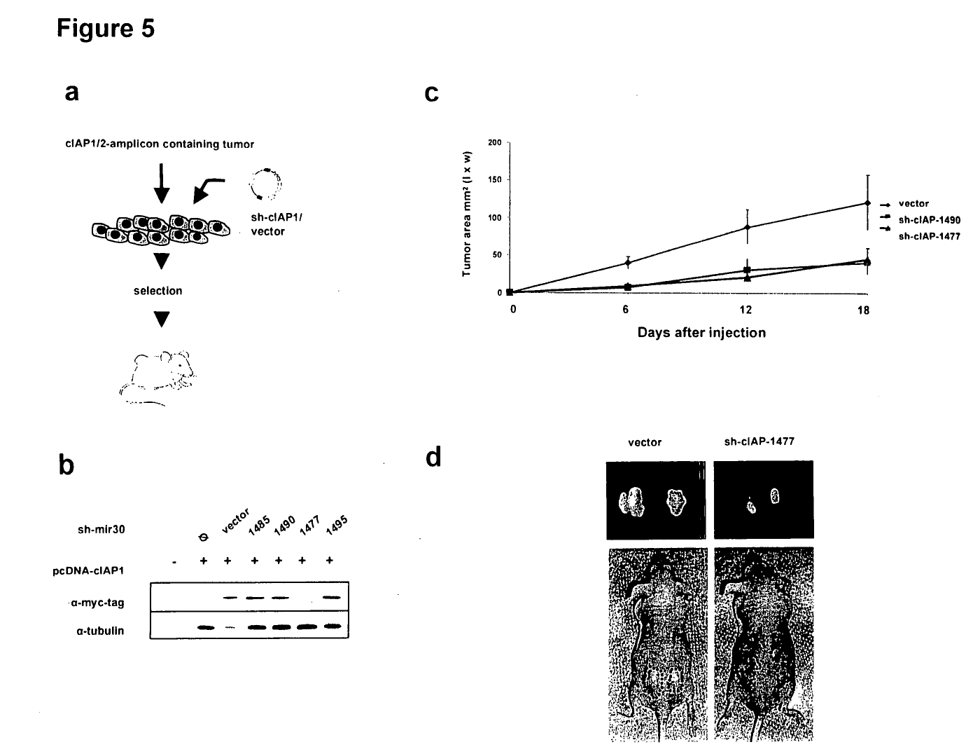

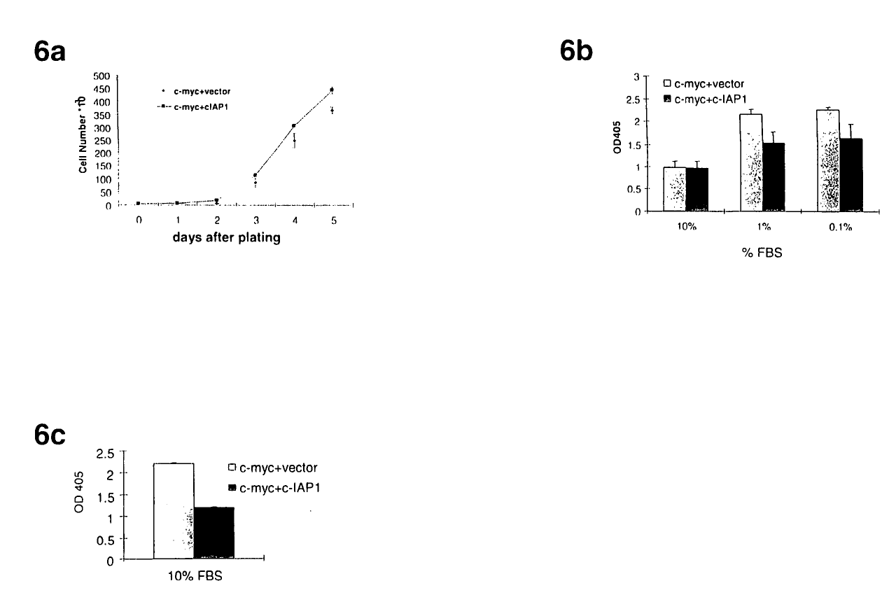

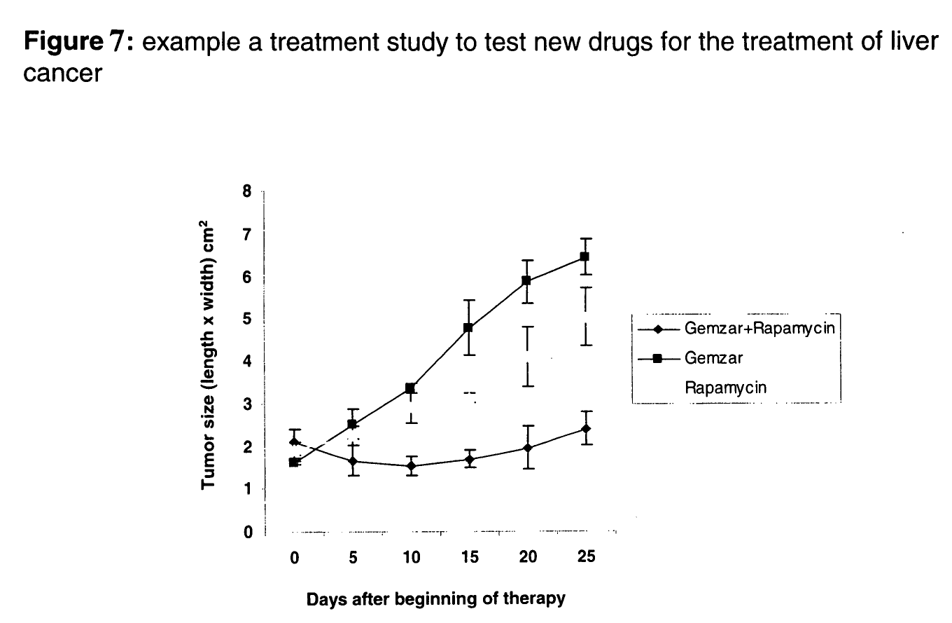

Most preferably, the therapies are used and evaluated in combination. For example, as shown in FIG. 7, upon onset of liver cancer tumors, animals can be treated with Gemcitabine (“Gemzar”), a chemotherapeutic agent that is an antimetabolite that functions as a mild chemotherapeutic to interfere with the growth of cancer cells. As shown in FIG. 7, it has virtually no effect on tumor growth of the particular tumor tested in FIG. 7. The tumor embodied in FIG. 2 can also be treated with Rapamycin, a targeted therapy that inhibits the mammalian target of rapamycin (mTOR). It has some effect on the tumor growth. In combination, however, as depicted in FIG. 7, the two therapies control tumor growth.

The size and growth of tumors after therapy can be monitored by a wide variety of ways known in the art. Preferably, whole body fluorescence imaging is used because the preferred viral vectors of this invention carry a GFP expression cassette. See, e.g, Schmitt et al., “Dissecting p53 tumor suppressor functions in vivo,” Cancer Cell 1:289-98 (2002). Tumors can also be examined histologically. Paraffin embedded tumor sections can be used to perform immunohistochemistry for cytokeratins and ki-67 as well as TUNEL-staining. The apoptotic rate of hepatocytes can be analyzed by TUNEL assay according to published protocols. Di Cristofano et al., “Pten and p27KIP1 cooperate in prostate cancer tumor suppression in the mouse,” Nature Genetics, 27:222-224 (2001).

Beyond having important implications for understanding liver cancer, the evaluations and observations made possible by the methods and models of this invention provide insight into the utility of targeted approaches in cancer therapy.

Throughout this specification and claims, the word “comprise” or variations such as “comprises” or “comprising”, will be understood to imply the inclusion of a stated integer or group of integers but not the exclusion of any other integer or group of integers.

### EXAMPLE 1

Generation and Transplantation of Genetically Altered Liver Progenitor Cells

To determine whether genetically modified hepatoblasts could colonize recipient livers, a protocol was used that optimizes engraftment of transplanted cells in the recipient liver. Embryonic hepatoblasts express high E-Cadherin levels on their cell surface, which enables these cells to be isolated to high purity from fetal livers using magnetic bead selection. (Nitou et al. “Purification of fetal mouse hepatoblasts by magnetic beads coated with monoclonal anti-e-cadherin antibodies and their in vitro culture.” Exp. Cell Res. 279, 330-343. (2002)). These cells express markers characteristic of bi-potential oval cells, the presumed cellular target of transformation in the adult rodent liver.

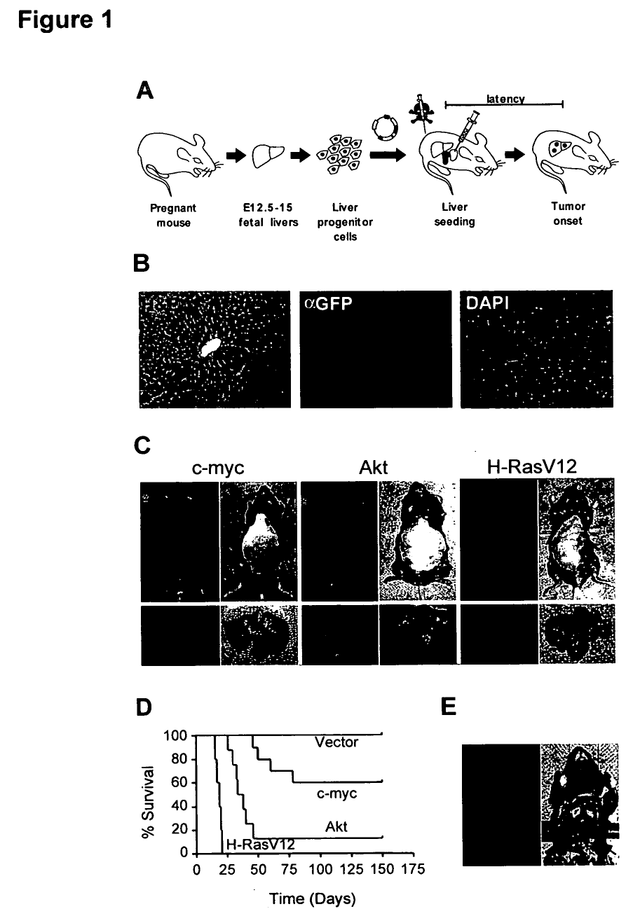

Animals were pretreated with retrorsine, an alkaloid that exerts a strong and persistent block of native hepatocyte proliferation and increases the competitive advantage of transplanted cells. Ten days after the last retrorsine treatment, 2×10⁶ GFP-tagged E-Cadherin⁺ liver progenitor cells were delivered to the liver by intrasplenic injection. Using this protocol, one week after injection approximately one percent of the host liver consisted of “seeded” GFP-positive cells that were embedded within the normal liver architecture (FIG. 1C).

Generation of Liver Carcinomas from Transplanted Liver Progenitor Cells

Hepatoblasts were isolated from p53−/−fetal livers and the cells were transduced with retroviruses co-expressing Myc (c-myc), activated Akt (Akt1), or oncogenic Ras (H-rasV12) (each of which affect signaling pathways altered in human liver cancer) and a GFP reporter. As above, these transduced cell populations were transplanted into retrorsine treated mice (see FIG. 1A). To further facilitate expansion of the transplanted cells, recipient mice were treated with CCI4 (Guo et al. “Liver repopulation after cell transplantation in mice treated with retrorsine and carbon tetrachloride.” Transplantation 73, 1818-1824. (2002)) and monitored for signs of disease by abdominal palpation of the liver and whole body fluorescence imaging. Although p53−/− hepatoblasts were not tumorigenic in our system, each of the cell populations that also expressed an oncogene eventually produced GFP-positive tumors in the livers of recipient mice (FIG. 1C, top).

Gross pathological analysis of explanted livers revealed that Myc-expressing tumors differ significantly from those expressing Akt or Ras (FIG. 1C, bottom). First, Myc-expressing tumors grow primarily as unilocular tumors, whereas Akt- and Ras-derived tumors show aggressive, multilocular and infiltrative intrahepatic growth. Second, the innate tumorigenicity of p53−/− liver progenitor cells expressing Myc was significantly lower than those expressing Akt or Ras; thus, Akt or Ras trigger the development of liver carcinomas with an efficiency of nearly 100%, while Myc produced tumors at a penetrance around 40% (FIG. 1D). In most instances, GFP-positive cells derived from these tumors could be readily grown in culture, and subsequently formed secondary tumors upon sub-cutaneous injection into immunocompromised mice or direct intrahepatic injection into syngeneic recipients (data not shown).

Murine Liver Carcinomas Histopathologically Resemble Features of Human HCC

To determine whether the murine tumors produced from liver progenitors resemble human liver cancer, a panel of hematoxylin/eosin (H&E) stained sections derived from primary Myc-induced murine hepatomas were examined by an experienced liver pathologist. These tumors were classified as moderately well to poorly differentiated HCCs with a mostly solid, sometimes mixed solid/trabecular growth pattern. A smaller proportion of tumors revealed growth patterns resembling trabecular or pseudoglandular HCC (data not shown). All tumors examined stained positive for cytokeratin 8, confirming they were derived from the liver lineage. Furthermore, transplanted tumors retained their HCC histology when injected orthotopically into the liver, or subcutaneously into immunocompromised mice (data not shown). These findings confirm that ex vivo manipulated liver progenitor cells can produce tumors that recapitulate the histopathology of human HCC.

ROMA Identifies Spontaneous Mutations in a Subset of Murine Liver Carcinomas

Epithelial cancers require a series of genetic alterations during clonal evolution to an advanced disease. To molecularly characterize the murine HCCs described above, spontaneously acquired lesions in those cancers were analyzed using representational oligonucleotide microarray analysis (ROMA), a genome-wide scanning method capable of identifying copy number alterations in tumor cells at high resolution (Lucito et al. “Representational oligonucleotide microarray analysis: a high-resolution method to detect genome copy number variation.” Genome Res. 13, 2291-2305. (2003)). Each human or mouse ROMA array consisted of 85K oligonucleotide probes designed to the UCSC Apr/2003 draft assembly of human genome and the UCSC Feb/2003 mouse genome, respectively, allowing genome scanning at a theoretical resolution of ˜35 kb.

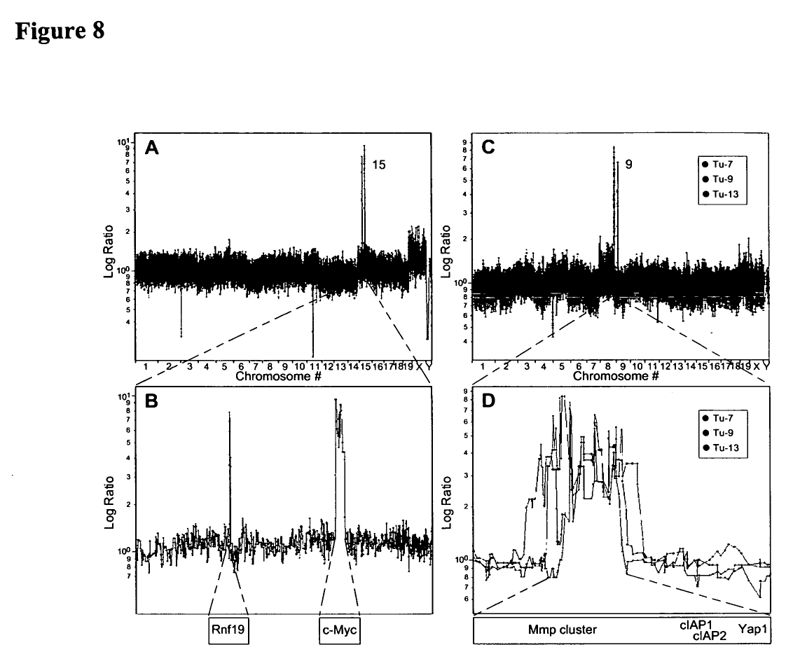

Genomic representations were produced from DNA obtained from several murine liver tumors and from normal mice tissue of the same genetic background, fluorescently labeled and hybridized to microarrays. The data derived after scanning was normalized as described (Sebat et al. “Large-scale copy number polymorphism in the human genome.” Science 305, 525-528. (2004)). Even though all tumors were derived from cells harboring two defined genetic lesions, some displayed a small number of focal copy number alterations. For example, a ras-expressing tumor harbored two focal amplifications on chromosome 15 (FIG. 8A), including a 250 Kb amplicon that contains Rnf19 and a 2 Mb amplicon containing c-myc (FIG. 8B). While Rnf19 has not been previously linked to tumorigenesis, c-myc amplification is a common event in human liver cancer and, furthermore, c-myc cooperates with oncogenic ras in transgenic models of HCC (Sandgren et al. “Oncogene-induced liver neoplasia in transgenic mice.” Oncogene 4, 715-724. (1989)). That a mutation affecting an established liver oncogene can occur spontaneously in these tumors underscores the relevance of the model of the present invention, and suggests that further analyses would reveal other genes involved in human cancer.

Recurrent Amplification of Chromosome 9 μL in Myc-Expressing HCCs

ROMA was also performed on seven independently derived Myc-expressing HCCs, and identified a focal amplicon on mouse chromosome 9qA1 in three of these tumors (FIG. 8C). As shown in a high resolution view (FIG. 8D), the minimal overlapping region was approximately 1 Mb and contained genes encoding for several matrix metalloproteinases (MMPs), Yap1, c1AP1 (Birc2), and c1AP2 (Birc3) as annotated in the UCSC genome browser. An EST to Porimin also maps to this region. Amplification of this region was confirmed by genomic Q-PCR using a probe targeting the middle of the 9qA1 amplicon within the c1AP1 gene, a technique that also identified the 9qA1 amplicon in a fourth myc-expressing tumor (data not shown). Remarkably, 9qA1 was never found amplified in 15 other liver carcinomas expressing either Ras or Akt. These observations suggest that at least one of the genes in the 9qA1 region cooperates with myc and p53 loss to promote hepatocarcinogenesis.

Comparative Oncogenomics Reveals Lesions in Common Between Murine and Human Cancers

In parallel to the analysis of murine HCCs, ROMA was conducted on 25 human HCC samples. These human tumors showed more complex alterations than the murine HCCs, yet we were able to detect copy number alterations affecting genes previously linked to HCC. For example, three tumors had a chromosome 11 amplification containing CCND1 (cyclin D1), two had a chromosome 7 amplification containing c-MET, and one had a deletion of chromosome 9 harboring the CDKN2A (INK4a/ARF) locus (data not shown).

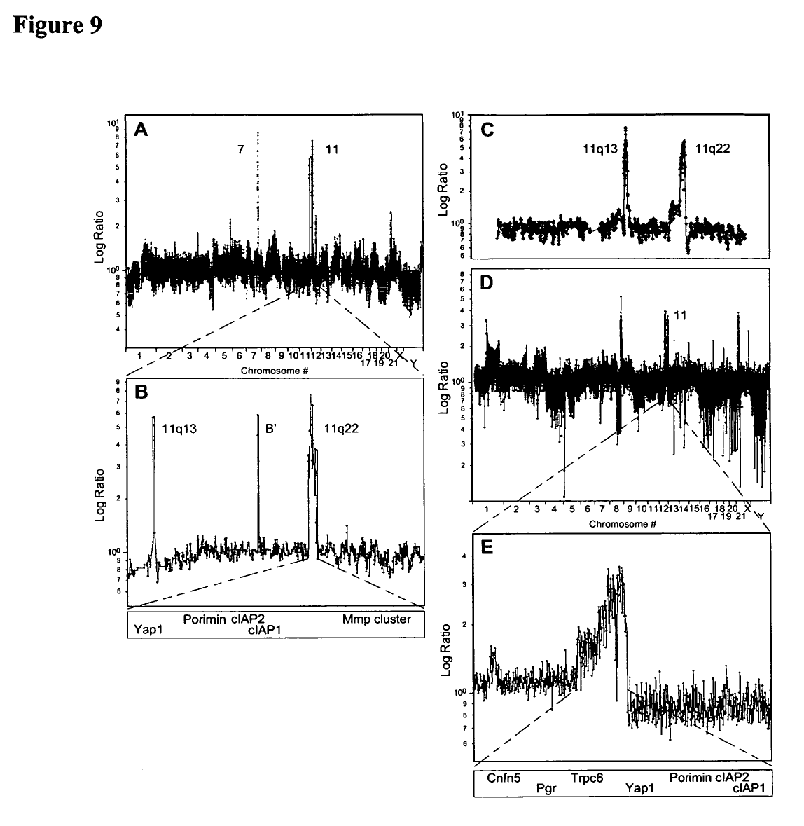

FIG. 9A shows a genome-wide profile of a liver tumor with a c-MET amplification (left peak) on chromosome 7, and three sharply delineated amplifications on chromosome 11 (FIG. 9B), including CCND1, B′ (containing no known genes), and 11q22. Only focal gains or losses <5 MB were included in the analysis. Interestingly, the amplified region of human chromosome 11q22 is syntenic to mouse 9qA1, the region amplified in murine HCCs described above. Through the use of ROMA and/or genomic quantitative PCR to the c1AP1 and c1AP2 loci, we detected this same amplicon in a panel of human esophageal cancers (FIG. 9C, 4 of 53 tested; data not shown), indicating that it occurred in gastrointestinal malignancies derived from developmentally related organs. Much like the chromosome 9 amplicon in murine HCCs, the boundaries of this 11q22 amplicon include genes encoding several matrix metalloproteinases, Porimin, Yap 1, c1AP1 and c1AP2.

The human 11q22 amplicon has previously been observed at low frequency in other human cancers, although no driver gene has been decisively identified. While it represents only one of many low frequency events in these tumors, our cross-species comparison suggests that a gene(s) in the amplified region is crucial for tumorigenesis in certain contexts. In an attempt to further narrow down potential candidates, a series of other cancers that were previously analyzed by ROMA were examined to identify the minimal region of overlap. FIGS. 9D and 9E show an example of an ovarian carcinoma harboring an 11q22 amplicon that was particularly informative: although this tumor is genomically unstable, the MMPs are excluded from this 11q22 amplification suggesting that overexpression of this cluster is not the key selected driving event. Therefore, the genes contained within the epicenter of the amplicon are Porimin, Yap1, c1AP1 and c1AP2.

c1AP1 is Consistently Overexpressed in Tumors Harboring the Murine 9qA1 and Human 11q22 Amplicons

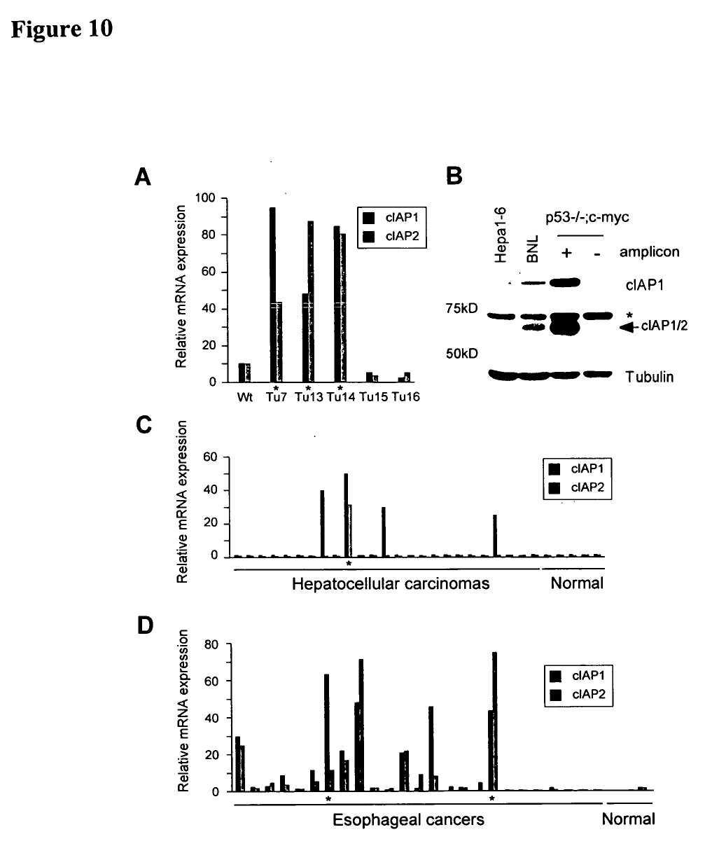

One criterion for establishing whether a gene in an amplicon might contribute to tumorigenesis is that it shows elevated expression in the tumor. RT-Q-PCR analysis was conducted on a series of tumors to examine which genes in the mouse 9qA1 and human 11q22 amplicons were consistently overexpressed. The murine p53−/−; myc HCCs that contain the 9qA1 amplicon consistently overexpressed c1AP1 and c1AP2 mRNA, and c1AP1 protein (FIGS. 10A and B). These genes were not upregulated in tumors without the amplicon. Both genes were overexpressed in the human HCC and esophageal tumors harboring the 11q22 amplicon, but also in a substantial number of tumors without c1AP1/c1AP2 copy number elevation (FIGS. 10C and D; 4 of 25 HCC; 15 of 50 esophageal). Interestingly, c1AP1 was the only c1AP overexpressed in human HCCs without 11q22 amplification (FIG. 10C), hinting that this gene might have a particularly important role in this disease. Based on these analyses, c1AP1 is a likely driver gene in liver cancer.

c1AP1 has Oncogenic Properties

Inhibitor of apoptosis (IAP) proteins were originally identified in baculovirus because of their potential to inhibit cell death of infected cells. Similar to their viral counterparts, overexpression of cellular 1APs can inhibit apoptosis induced by different stimuli. Although 1APs have been shown to bind and inhibit caspases, it is controversial as to whether they are important regulators of apoptosis in mammalian cells. Furthermore, although indirect evidence point towards 1APs playing a role in oncogenesis (Wright & Duckett. “Reawakening the cellular death program in neoplasia through the therapeutic blockade of IAP function.” J. Clin. Invest 115, 2673-2678. (2005)), there remains no direct evidence that these genes actively contribute to tumor initiation or maintenance.

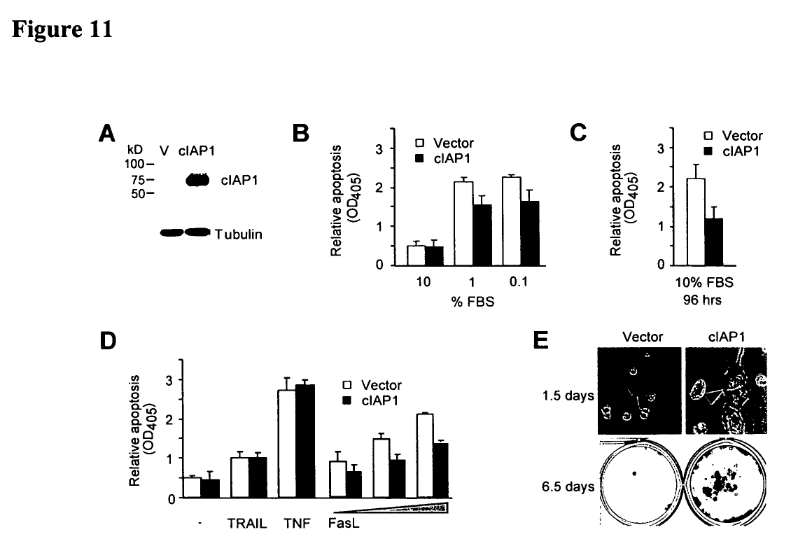

A significant advantage of profiling the genomes of defined murine tumors is that candidate genes can be validated in the genetic context, in which the mutation spontaneously arose during tumorigenesis. Our studies identified the 9qA1 amplicon in tumors derived from p53−/− hepatoblasts expressing Myc but not in other configurations, suggesting these cells would be ideal for evaluating the oncogenic properties of c1AP1. Therefore, p53−/−; myc liver progenitor cells expressing c1AP1 or a control vector were produced using retroviral mediated gene transfer, and the resulting cell populations were examined for transgene expression (FIG. 11A) and subjected to different apoptotic triggers. In this cell type, c1AP1 overexpression conferred a modest protection from growth factor withdrawal and spontaneous cell death at confluence (FIG. 11B and 11C). Surprisingly, c1AP1 had no effect on apoptosis induced by the death ligands TRAIL and TNFa (FIG. 11D), although it did confer substantial short and long-term protection from Fas-mediated apoptosis (FIGS. 11D and E). Thus, c1AP1 can suppress apoptosis in murine hepatoblasts in vitro.

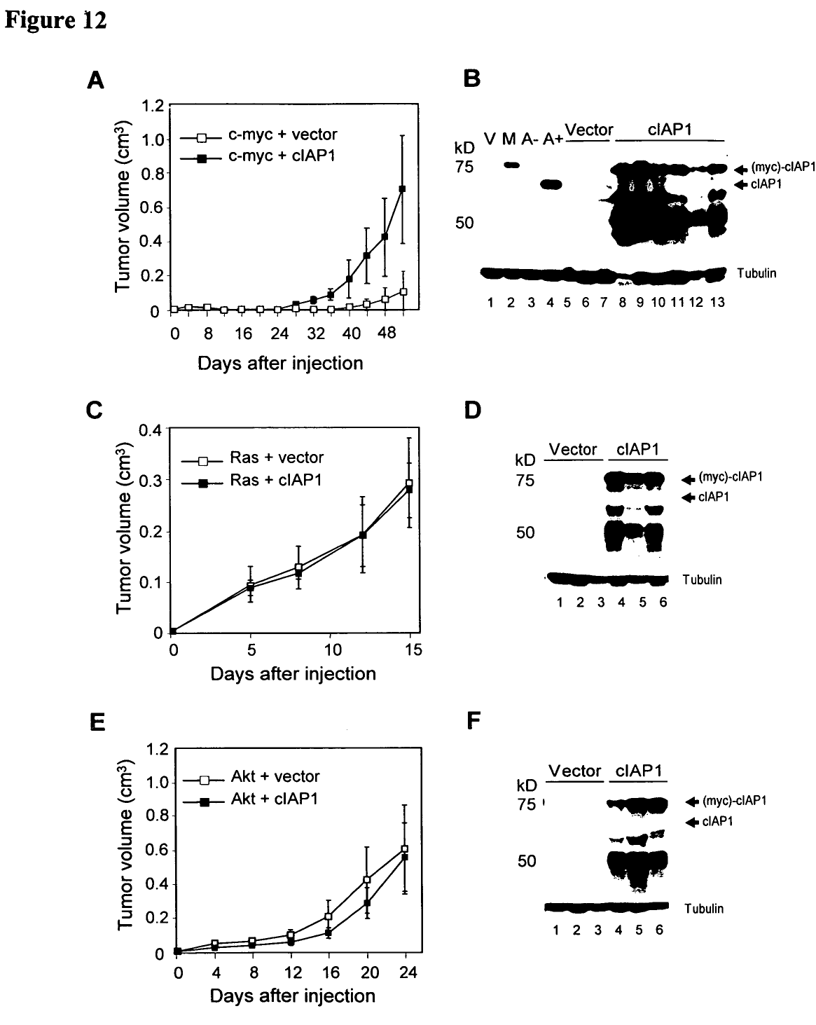

To determine whether c1AP1 could function as an oncogene in vivo, the hepatoblast cultures described above were injected subcutaneously into nude mice to facilitate precise measurement of tumor growth. c1AP1 overexpression significantly accelerated the growth of p53−/− hepatoblasts expressing Myc (FIG. 12A) (onset time of 24±2.3 for myc+c1AP1 vs. 45±12.2 for myc+vector (p=0.02)). The tumors displayed the histopathology of moderately well to poorly differentiated HCC (data not shown) and stably overexpressed the c1AP1 protein at high levels (FIG. 12B, compare lane 2 to lanes 7-13). Also present were low molecular weight forms of c1AP1, consistent with the susceptibility of this protein to proteolytic degradation. Interestingly, one control tumor that was harvested at a very small size showed elevated levels of c1AP1 (FIG. 12B, lane 6), suggesting that a subset of these cells had acquired a spontaneous alteration that upregulated the gene.

The ability of c1AP1 to promote tumorigenicity in cooperation with Akt or Ras was also examined. Using the same procedures described above, we produced p53−/− hepatoblasts expressing either Akt or Ras with or without c1AP1. In contrast to the Myc configuration, overexpression of c1AP1 has no impact on the onset or progression of tumors expressing Akt or Ras (FIGS. 12C and E), even though c1AP1 was efficiently expressed (FIGS. 12D and F). Thus, c1AP1 is selectively oncogenic in the genetic context where its amplification occurs.

c1AP1 and c1AP2 are Required for Rapid Tumor Growth

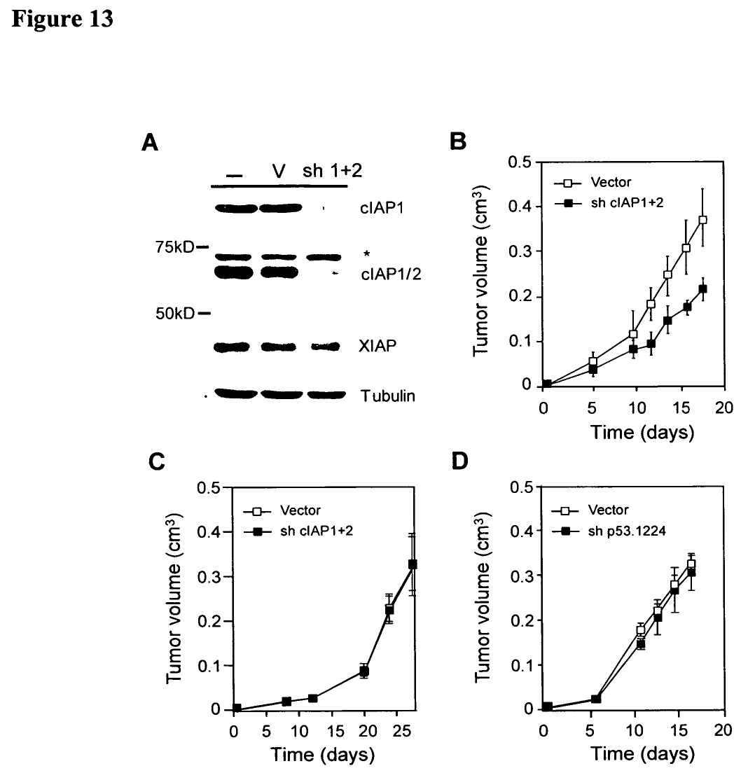

The above data demonstrates that c1AP1 can causally contribute to HCC development. To determine whether the c1AP proteins were required to sustain tumor growth, the impact of reducing c1AP levels on the growth of Myc-induced HCCs was examined in vivo. The expression of c1AP1 and c1AP2 was suppressed, since c1AP2 can be upregulated in response to c1AP1 suppression. First, a series of retroviral vectors expressing shRNAs capable of suppressing c1AP1 (hygromycin selectable) and c1AP2 (puromycin selectable) expression by RNA interference were generated. The best performing shRNAs were co-introduced into outgrown Myc-induced HCC cells containing or lacking the 9qA1 amplicon. Using these vectors, there was significant downregulation of endogenous c1AP1/2, as shown by immunoblotting using an antibody directed against c1AP1 or an antibody that cross-reacts with both c1AP1 and c1AP2 (FIG. 13). Some of these cells were also subsequently injected subcutaneously into the flanks of immunocompromised mice, and tumor growth was assessed by caliper measurement.

In tumors harboring the 9qA1 amplicon, suppression of c1AP1/2 had a marked impact on tumor growth. Thus, tumors expressing c1AP1 and c1AP2 shRNAs showed a reduced growth rate compared to parallel tumors expressing the control vectors (FIG. 13B). By contrast, these same shRNAs had no impact on the growth of an amplicon negative tumor derived from the same genotype (FIG. 13C), suggesting that only cells selected for c1AP overexpression are sensitive to c1AP inhibition. This latter observation also rules out off-target effects of these shRNAs on tumor growth. Consistent with this hypothesis, a p53 shRNA did not inhibit the growth of the p53−/−; myc tumor containing the 9qA1 amplicon (FIG. 13D). Therefore, c1AP1 and 2 are required for the efficient growth of tumors harboring the 9qA1 amplicon and thus may be therapeutic targets in a subset of human cancers.

A description of certain preferred claims of the invention follows.

What is claimed is:

1. A method for making a non-human liver cancer model, said method comprising:
(a) altering hepatocytes to increase oncogene expression, to reduce tumor suppressor gene expression or both, and
(b) transplanting said hepatocytes into a recipient non-human animal wherein the hepatocytes engraft the liver of said animal and a liver cancer tumor develops from at least one of the altered hepatocytes.

2. A method for making a non-human liver cancer model, said method comprising:
(a) altering hepatocytes to increase oncogene expression, to reduce tumor suppressor gene expression or both, and
(b) transplanting said hepatocytes subcutaneously into a recipient non-human animal and a hepatocellular cancer tumor develops from at least one of the altered hepatocytes.

3. The method of claim 1 or 2 wherein the hepatocytes are altered to increase oncogene expression by transduction with an oncogene.

4. The method of claim 3 wherein the oncogene is the H-ras oncogene.

5. The method of claim 1 or 2 wherein the hepatocytes are altered to reduce tumor suppressor gene expression by transduction with a short hairpin RNA against a tumor suppressor gene.

6. The method of claim 5 wherein the short hairpin RNA is directed against p53.

7. The method of claim 1 or 2 wherein the hepatocytes are embryonic or primary hepatocytes.

8. The method of claim 1 or 2 wherein tumor onset or growth is monitored by fluorescence imaging using a marker gene.

9. The method of claim 8 wherein the marker gene is GFP.

10. The method of claim 3 or 5 wherein the transduction occurs with a gene operatively linked to a ubiquitous promoter.

11. The method of claim 1 wherein the altered hepatocytes are transplanted into the spleen of a recipient non-human animal and said hepatocytes migrate via the portal vein into the recipient liver.

12. The method of claim 11 wherein the recipient non-human animal is pretreated with a liver cell cycle inhibitor.

13. The method of claim 12 wherein the liver cell cycle inhibitor is Retrorsine.

14. The method of claim 11 wherein the recipient non-human animal is post treated by several administrations of CCl₄.

15. A method for determining the efficacy of a therapy in treating liver cancer, comprising:
(a) administering to a non-human animal, produced by the method of claim 1, the therapy, and
(b) monitoring the non-human animal for liver tumor formation or growth, wherein increased time to tumor formation or growth indicates sensitivity of the tumor to the therapy.

16. The method of claim 15 further comprising the steps, after step (b), of:
(c) administering the therapy to the non-human animal which causes remission; and
(d) monitoring the length of time until relapse, wherein increased time of secondary tumor formation indicates sensitivity of the tumor to the therapy.

17. The method of claim 15 or 16 wherein the effect of the therapy is assessed by monitoring fluorescence imaging to track tumor formation or growth.

18. The method of any one of claims 15-17 wherein the hepatocyte is altered by an overproduction of an oncoprotein and the therapy is an inhibitor of the oncoprotein.

19. The method of any one of claims 15-17 wherein the hepatocyte is altered by an underproduction of a tumor suppressor gene and the therapy is an agonist of the tumor suppressor gene.

20. The method of claim 18 or 19 wherein the therapy is administered with a drug that induces apoptosis of proliferating hepatocytes.

21. A non-human animal produced by the method of claim 1 or 2.

22. A method for identifying a gene that is involved in liver cancer, by introducing into a non-human animal an altered hepatocyte comprising a nucleic acid construct encoding a short hairpin RNA, wherein the short hairpin RNA is complementary to at least a portion of the gene, wherein the altered hepatocyte exhibits decreased expression of the gene, and wherein the altered hepatocyte gives rise to a transfected tumor cell in vivo.

23. A method for identifying a gene that is involved in liver cancer, by producing a genetic profile of the cancer and identifying those genes whose copy number has been altered in the cancer.

24. The method of claim 15, wherein the therapy normalizes the genetic profile in the context of at least one of the genes whose copy number has been altered in the cancer.

25. The method of claim 24, wherein the therapy is a short hairpin RNA that knocks down the copy number of at least one gene whose copy number has been increased in the cancer.

26. The method of claim 25, wherein the gene is c-1AP-1 or c-1AP2.
